(19) United States
(12) Plant Patent Application Publication
(10) Pub. No.: US 2005/0223449 P1
(43) Pub. Date: Oct. 6, 2005

(54) Senecio plant named 'Sunsenebabu'

(50) SENECIO CRUENTUS X SENECIO HERITIERI; SENECIO

(76) Inventor: Kiyoshi Miyazaki, Hikone (JP)

Correspondence Address: CHRISTIE, PARKER & HALE, LLP, PO BOX 7068, PASADENA, CA, 91109-7068, US

(21) Appl. No.: 10/814,612

(22) Filed: Mar. 30, 2004

(51) Int. Cl. A01H 5/00

(52) U.S. Cl. PLT/263

(57) ABSTRACT

Disclosed herein is a new variety of Senecio plant having a semi-dwarf, obconical shaped with abundant branching and small leaves. The flowers are single and small, the petals having a vivid violet color and the disk color is brilliant violet. The blooming time is early and flowering duration is longer than Senecio cruentus. ‘Sunsenebabu’ has low fertility.

Botanical/commercial classification:

Senecio cruentus×Senecio heritierii/Senecio Plant

Varietal denomination: cv. Sunsenebabu

### BACKGROUND OF THE VARIETY

The present invention relates to a new variety of Senecio plant originated from crossing of a cultivar ‘Extra Blue’ as the female parent and a variety of Senecio heritieri as the male parent.

There are many varieties in Senecio L. and Senecio cruentus well known as ‘Cineraria’ and cultivated in the world. There are many cultivated varieties with flowers of a single color of white, pink red, blue or violet. Some varieties have marginal variegation with off color parts.

Progress

The female parent ‘Extra Blue’ used in the crossing of ‘Sunsenebabu’ is a cultiver of Senecio cruentus. It is early flowering variety having dwarf and mounding shape with large leaves. It has small single flowers, the petals having a vivid purple color. The seed of ‘Extra Blue’ is commercially available.

The male parent Senecio heritieri used in the crossing of ‘Sunsenebabu’ is a cultiver, having a high and dome-shaped growth habit with abundant branching with small leaves. It has small single flowers, the petals having strong purple with vague white center coloration. Senecio heritieri was introduced from nurseries in England, has no variety name and is not patented nor sold in the United States.

In January 1996, crossing of ‘Extra Blue’ as the female parent and Senecio heritieri as the male parent was conducted at Hakushu-cho, Kitakoma-gun, Yamanashi-ken, Japan. The seedlings obtained from that crossing were grown in pots in glasshouses and evaluated from July 1996. One seedling was selected in view of its growth habit, flower color and flowering time in December 1996. That seedling was propagated by cutting and a trial was carried out by flower potting from July 1999, at Hakushu-cho, Kitakoma-gun, Yamanashi-ken, Japan. The botanical characteristics of that plant were then examined, using similar varieties ‘Sunsenebu’ (U.S. Plant Pat. No. 12,104) and ‘Miss Yokohama’ for comparison. As a result, it was concluded that this Senecio plant is distinguishable from any other variety, whose existence is known to us, and is uniform and stable in its characteristics. Then the new variety of Senecio plant was named ‘Sunsenebabu’.

In the following description, the color-coding is in accordance with the Horticultural Colour Chart of The Royal Horticultural Society, London, England (R.H.S. Colour Chart).

### SUMMARY OF THE VARIETY

This new variety is unlike any Senecio commercially available as evidenced by the following unique combinations of characteristics.

1. Semi-dwarf, obconical plant shape having abundant branching with small leaves.

2. The flowers are single and small. The petal color is vivid violet (R.H.S. 94A). The disk color is brilliant violet (R.H.S. 89D).

3. Blooming time is early, and flowering duration is long.

4. Having low fertility.

The new variety ‘Sunsenebabu’ differs from the similar variety ‘Sunsenebu’ in the following points.

1. The plant size of ‘Sunsenebabu’ is smaller than that of ‘Sunsenebu’.

2. The flower diameter of ‘Sunsenebabu’ is smaller than that of ‘Sunsenebu’.

3. The disk color of ‘Sunsenebabu’ is brilliant violet (R.H.S. 89D). That of ‘Sunsenebu’ is deep violet (R.H.S. 93A).

4. The peduncle length of ‘Sunsenebabu’ is shorter than that of ‘Sunsenebu’.

5. The blooming time of ‘Sunsenebabu’ is earlier than that of ‘Sunsenebu’.

The new variety ‘Sunsenebabu’ differs from the similar variety ‘Miss Yokohama’ in the following points.

1. The plant height of ‘Sunsenebabu’ is higher than that of ‘Miss Yokohama’.

2. Number of the branches of ‘Snusenebabu’ is more than that of ‘Miss Yokohama’.

3. The leaf of ‘Sunsenebabu’ is smaller than that of ‘Miss Yokohama’.

4. The disk color of ‘Sunsenebabu’ is brilliant violet (R.H.S. 89D). That of ‘Miss Yokohama’ is vivid violet (R.H.S. 96A).

5. The blooming time of ‘Sunsenebabu’ is earlier than that of ‘Miss Yokohama’.

6. The flowering duration of ‘Sunsenebabu’ is longer than that of ‘Miss Yokohama’.

This new variety of Senecio Plant ‘Sunsenebabu’ was asexually reproduced by the use of cuttings at Hakushu-cho, Kitakoma-gun, Yamanashi-ken, Japan, and homogeneity and stability thereof were confirmed. The instant plant retains its distinctive characteristics and reproduces true to type in successive generations.

### BRIEF DESCRIPTION OF THE PHOTOGRAPH

The depicted plants had been reproduced by the use of cuttings and were photographed during January 2003 while growing outdoors in 12 cm pots at an age of approximately 5 months at Yokaichi-shi, Shiga-ken, Japan.

FIG. 1 illustrates a typical plant of the new variety of Senecio plant ‘Sunsenebabu’ growing in a pot.

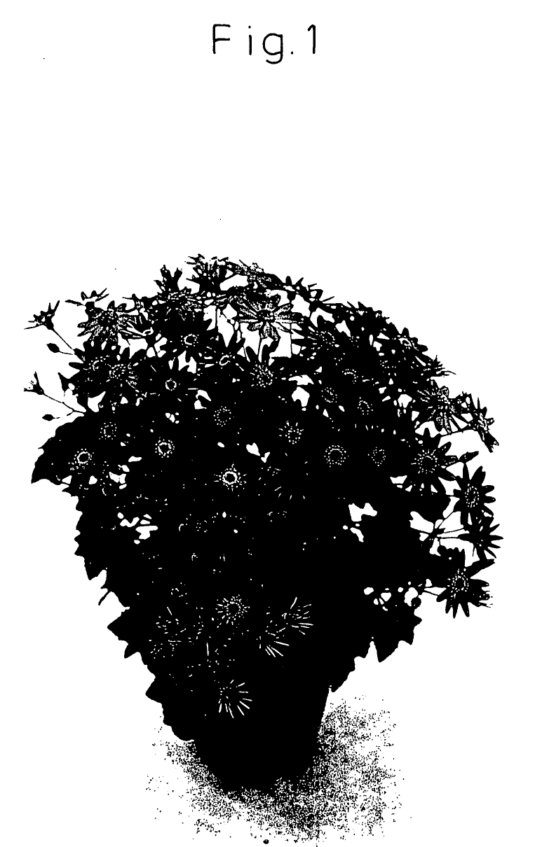

FIG. 2 illustrates a close-up view of typical blossoms of the new variety of Senecio plant ‘Sunsenebabu’.

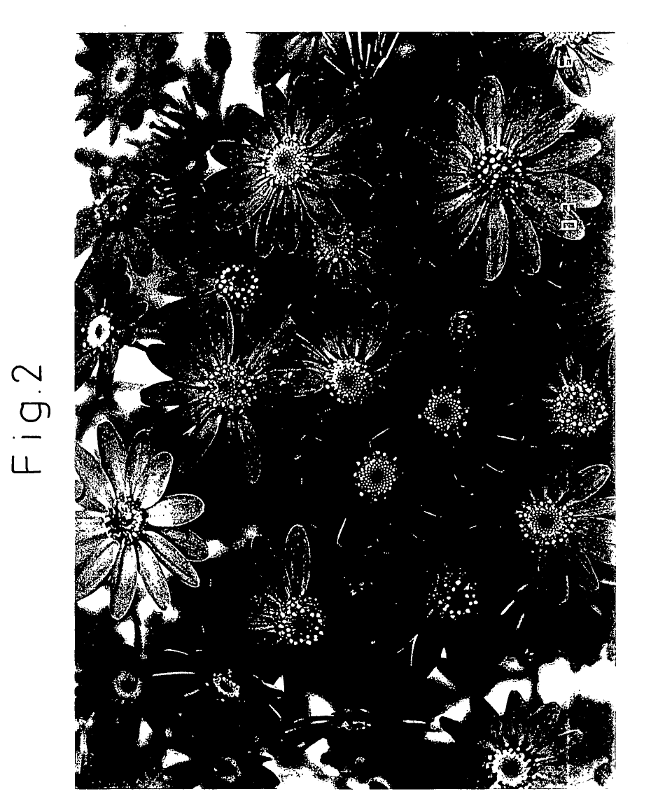

### DESCRIPTION OF THE VARIETY

The botanical characteristics of the new and distinct variety of Senecio plant named ‘Sunsenebabu’ are as follows when observed during January at Yokaichi-shi, Shiga-ken, Japan, at an age of approximately 5 months.
Plant:
   Growth habit.—Semi-dwarf, obconical.
   Height.—Approximately 27 cm.
   Width.—Approximately 18 cm.
Stem:
   Thickness.—Approximately 3.5 mm.
   Color.—R.H.S. 139C (moderate yellow green).
   Anthocyanin coloration.—Present in parts of the stem, e.g. can be seen at the part of peduncle.
   Branching.—Abundant.
   Type of primary lateral shoot.—Branch from every node.
   Pubescence.—Dense.
   Length of internode.—Approximately 1.5 cm.
Leaf:
   Whole shape.—Cordate.
   Leaf margin.—Dentate.
   Apex shape.—Obtuse.
   Base shape.—Cordate.
   Degree of undulation.—Weak.
   Length.—Approximately 6.8 cm.
   Width.—Approximately 7.8 cm.
   Diameter of petiole.—Approximately 3.4 mm.
   Length of petiole.—Approximately 5.5 cm.
   Color of upper surface.—R.H.S. 146A (Moderate olive green).
   Color of reverse surface.—R.H.S. 191B (pale yellow green).
   Anthocyanin coloration of reverse surface.—Absent.
   Pubescence of upper surface.—Moderate.
   Pubescence of reverse surface.—Dense.
Flower:
   Type of flower.—Single.
   Shape of flower cluster.—Flat.
   Diameter of flower cluster.—Approximately 31 cm.
   Height of flower cluster.—Approximately 19 cm.
   Transected shape of corolla.—Flat.
   Diameter of flower.—Approximately 4.4 cm.
   Diameter of disk.—Approximately 1.1 cm.
   Color of petal.—R.H.S. 94A (vivid violet).
   Marginal variegation.—Absent.
   Color of disk flower.—R.H.S. 89D (brilliant violet).
   Petal length.—Approximately 1.8 cm.
   Petal width.—Approximately 0.6 cm.
   Shape of petal.—Oblong.
   Lengthwise warp of petal.—Flat.
   Shape of petal tip.—Acute.
   Number of ray flowers.—13.
   Number of disk flowers.—Approximately 110.
   Diameter of pedicel.—Approximately 1.1 mm.
   Length of pedicel.—Approximately 4.1 cm.
   Number of flowers per flower cluster.—Abundunt.
   Scent.—Present.
Involucre:
   Type.—Bracts in a whorl, fused at the base, not recurred.
   Length of bracts (separated portion).—Approximately 1.1 mm.
   Width of bracts (separated portion).—Approximately 1.0 mm.
   Apex shape of bracts.—Acute.
   Color.—R.H.S. 144B (strong yellow green).
   Anthocyanin coloration.—Absent.
Pistil:
   Color.—R.H.S. 86B (strong purple).
   Number.—1.
   Type.—Style branches truncate.
Stamen:
   Color.—R.H.S. 86B (strong purple).
   Type.—Synantherous.
   Blooming time.—Begining of November (cutting in July).
Hardiness:
   Cold.—Good.
   Heat.—Good.
Resistance:
   Disease.—Good.
   Insect.—Good.

The new variety and Senecio cruentus have similar resistance to powdery mildew, leaf spot, aphid, whitefly and thrips. The new variety, ‘Sunsenebabu’, is most suitable for flower potting.

1. A new variety of Senecio plant named ‘Sunsenebabu’, substantially as herein illustrated and described.
